(19) United States
(12) Patent Application Publication
(10) Pub. No.: US 2008/0132990 A1
(43) Pub. Date: Jun. 5, 2008

(54) STENT DELIVERY SYSTEM WITH IMAGING CAPABILITY

(75) Inventor: M. Kevin Richardson, Hopkinton, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SCIMED LIFE SYSTEMS, INC., Maple Grove, MN (US)

(21) Appl. No.: 12/029,959

(22) Filed: Feb. 12, 2008

Related U.S. Application Data
(63) Continuation of application No. 10/856,260, filed on May 27, 2004.

(51) Int. Cl. A61F 2/84

(52) U.S. Cl. 623/1.12; 623/1.11

(57) ABSTRACT

A stent delivery system includes a catheter shaft defining two lumens, for respectively receiving a guidewire and a fiber optic cable having a viewing capability. Specifically, the fiber optic cable has a first (e.g., proximal) end and a second (e.g., distal) end, and is adapted for transmitting illumination light from its first end to its second end while transmitting an image from its second end to its first end. The system further includes a stent positioned over the catheter shaft, and may also include means for deploying the stent. The stent may be of a self-expanding type or of an inflation type. The fiber optic cable is used to visually inspect proper deployment of the stent before, during, and after the stent deployment.

### CROSS-REFERENCE TO RELATED APPLICATION

This application is a continuation of U.S. application Ser. No. 10/856,260, filed May 27, 2004, the disclosure of which is incorporated by reference herein.

### FIELD OF THE INVENTION

The present invention relates to medical devices, and in particular to a stent delivery system adapted for advancing a guidewire and a fiber optic cable having an imaging capability.

### BACKGROUND OF THE INVENTION

Stents and stent delivery assemblies are utilized in a number of medical procedures and situations, and as such their structure and function are well known. A stent is a generally cylindrical prosthesis that is introduced via a catheter into a lumen of a body cavity or vessel. The stent is introduced into the cavity or vessel with a generally reduced diameter and then is expanded to the diameter of the cavity or vessel. In its expanded configuration, the stent supports and reinforces the cavity/vessel walls while maintaining the cavity/vessel in an open, unobstructed condition.

Both self-expanding and inflation (as by a balloon) expandable stents are well known and widely available. Self-expanding stents must be maintained under positive external pressure in order to maintain their reduced diameter configuration during delivery of the stent to its deployment site. Inflation expandable stents (also known as balloon expandable stents) are generally crimped to their reduced diameter about the delivery catheter, positioned at the deployment site, and then expanded to the cavity/vessel diameter by fluid inflation of the balloon positioned between the stent and the delivery catheter. Some examples of stents and stent delivery catheters are disclosed in co-assigned U.S. Pat. Nos. 6,626,934 and 6,620,122, which are incorporated by reference herein.

A stent delivery catheter is typically delivered over a guidewire. A guidewire is very flexible and has a smaller diameter than a stent delivery catheter, and therefore is inserted into the body cavity or vessel of interest first, over and along which a stent delivery catheter can follow. Typically, when applying a stent in a body cavity of interest, a guidewire is introduced into the body cavity through a working lumen defined in an endoscope. A physician advances an endoscope and the guidewire removably received therethrough into the body cavity of interest while observing an image received from the distal end of the endoscope. Once the distal end of the guidewire reaches the position of interest, as observed by the endoscope, the endoscope is withdrawn, leaving the guidewire in place. Thereafter, a stent delivery catheter is passed over the guidewire and the stent is deployed. To observe and ensure proper deployment of the stent, the endoscope is sometimes passed along the side of the stent during deployment. In addition, for example when applying a stent in a blood vessel, fluoroscopy (x-ray imaging of a moving object) is often used to ensure proper placement and deployment of the stent, as well known in the art.

An endoscope, however, has a diameter that is relatively large with respect to the body cavity or body lumen of interest. Thus, the use of an endoscope to deliver a guidewire (and hence a stent delivery catheter) becomes difficult in some applications. Furthermore, positioning an endoscope along the side of a stent to observe its proper deployment requires an even larger space, which is not always available. Still further, use of fluoroscopy to confirm proper positioning of a guidewire and/or a stent is a relatively cumbersome procedure and requires additional safety mechanisms for the patients as well as the doctors and their assistants.

A need exists for a stent delivery system having imaging (or viewing) capabilities that does not require the use of fluoroscopy or a relatively larger-diameter endoscope.

### SUMMARY OF THE INVENTION

To overcome the foregoing disadvantages, the present invention offers a double-lumen stent delivery system. The system includes a catheter shaft defining at least two lumens, for respectively receiving a guidewire and a fiber optic cable having a viewing capability. Specifically, the fiber optic cable has a first (e.g., proximal) end and a second (e.g., distal) end, and is configured to transmit illumination light from its first end to its second end while transmitting an image from its second end to its first end. In accordance with one aspect of the present invention, the diameter of the fiber optic cable is less than 1 mm.

The system further includes a stent positioned over the catheter shaft, and may also include means for deploying the stent. A stent may be applied in various systems of a patient including, but not limited to, GI (gastrointestinal), URO (urogenital), biliary, and vascular systems. The stent may be of the self-expanding type, and in such a case the means for deploying the stent include a proximally retractable sleeve coaxially placed over the stent to maintain the stent in a compressed state during delivery. Alternatively, the stent may be of the inflation type, and the means for deploying the stent include an inflatable balloon positioned between the catheter shaft and the stent.

In operation, a physician can advance the guidewire into the body cavity or vessel of a patient to a desired position, while visually observing the advancement of the guidewire using the fiber optic cable. The fiber optic cable can be used to visually locate and/or measure a stricture at which the stent is to be deployed. Once the guidewire is properly placed, in reliance on the image received from the fiber optic cable, the catheter shaft is passed along the guidewire to properly place the stent relative to the stricture. Then, the stent is deployed. The fiber optic cable can be used to observe proper deployment of the stent before, during, and after the deployment procedure. The fiber optic cable can additionally be used to observe tissue or lesion in the area of stent deployment. Further additionally, the fiber optic cable may be configured to transmit electromagnetic energy (including both visible and non-visible ranges) for further diagnosis/treatment purposes.

In accordance with another embodiment of the present invention, a stent delivery system includes a catheter shaft defining a lumen for removably receiving a fiber optic cable therethrough. The catheter shaft further defines a guide which extends axially along at least a portion of the axial length of the catheter shaft. The guide may have a generally C-shaped (or U-shaped) cross section so as to generally contain, but not necessarily constrain, a guidewire therethrough. In one embodiment, the overall cross section of the catheter shaft, defining both the lumen and the guide, is generally circular. The stent delivery system further includes a stent positioned over the catheter shaft, and may further include means for deploying the stent. The operation of the stent delivery system is generally the same as the first embodiment, except that the guidewire in this embodiment is placed within the guide.

In accordance with yet another embodiment of the present invention, a stent delivery system includes a catheter shaft defining a lumen for removably receiving a guidewire therethrough, and a fiber optic cable that is provided independently of the catheter shaft. As before, the stent delivery system further includes a stent positioned over the catheter shaft, and may further include means for deploying the stent. The operation of the stent delivery system is generally the same as the first embodiment, except that the fiber optic cable is placed and advanced independently of the catheter shaft including the guidewire.

According to the present invention, various embodiments of a stent delivery system adapted to accommodate both a guidewire and a small-diameter fiber optic cable are provided. The use of a fiber optic cable with an imaging capability permits a physician to visually observe not only the proper advancement and placement of the guidewire but also the proper deployment of a stent before, during, and after the deployment procedure. Thus, the present invention provides a compact stent delivery system, which reduces the need for using fluoroscopy or a relatively larger-diameter endoscope to deliver and deploy a stent.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a side view of the distal portion of an inflation-type stent delivery system formed according to one embodiment of the present invention;

FIG. 2 is an enlarged, partially cross-sectional, schematic view of the distal portion of the system of FIG. 1 (indicated by dashed circle 2 in FIG. 1);

FIG. 3 is a cross-sectional view of the system of FIG. 2;

FIG. 4 is a longitudinal cross-sectional view of a fiber optic cable suitable for use in a stent delivery system in accordance with the present invention;

FIGS. 4A and 4B are cross-sectional views taken along lines A-A and B-B, respectively, of the fiber optic cable of FIG. 4;

FIG. 5 is a partially schematic side view of a self-expanding type stent delivery system formed according to one embodiment of the present invention;

FIG. 6 is an enlarged, cross-sectional view of the distal portion of the system of FIG. 5;

FIG. 7 is a cross-sectional view of the system of FIG. 6;

FIG. 8 is a cross-sectional view of another embodiment of a stent delivery system including a guide for receiving a guidewire (or a fiber optic cable) therein, formed according to the present invention;

FIGS. 8A and 8B are cross-sectional views of further alternative embodiments of a stent delivery system including a guide for receiving a guidewire (or a fiber optic cable) therein, formed according to the present invention;

FIG. 9 is a side view of yet another embodiment of a stent delivery system, in which a fiber optic cable is provided independently of a catheter shaft, formed according to the present invention; and

FIG. 10 is a schematic view of an elongate imaging device consisting of a signal cable and an image sensor, for use in place of a fiber optic cable, in accordance with the present invention.

### DETAILED DESCRIPTION OF THE PREFERRED EMBODIMENT

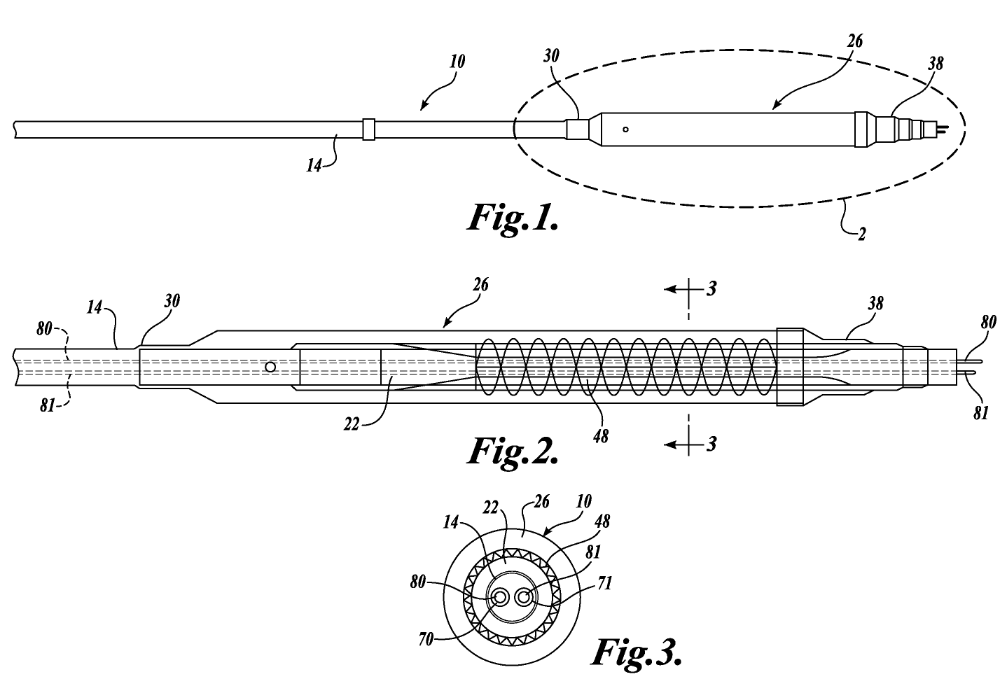

FIGS. 1-3 illustrate an inflation-type stent delivery system formed in accordance with the present invention. Referring to FIG. 1, a stent delivery system 10 has a catheter shaft 14 formed of any suitable flexible material, such as extruded plastic (e.g., polytetrafluoroethylene, polyether block amide, nylon, etc.). At the distal portion of the shaft 14 is disposed a sheath 26 coaxially surrounding the shaft 14. Referring additionally to FIG. 2, the distal portion of the shaft 14 is coupled to a balloon 22, which is constructed and arranged for expansion from a contracted state to an expanded state. The balloon 22 may be of any length depending on each application. The balloon 22 is shown in a folded, contracted state in FIG. 2. In use, the balloon 22 has a larger diameter which is obtained when the balloon 22 is expanded in any known manner. For example, the balloon 22 may be inflated by fluid (gas or liquid) from an inflation port (not shown) extending from an inflation lumen contained in the shaft 14 and opening into the balloon 22. Various means for inflating a balloon are well known in the art and thus need not be described in detail herein. A generally cylindrical stent 48 is mounted coaxially over the balloon 22. The sheath 26 is formed of a very flexible thin walled sleeve having a proximal end 30 and a distal cuff or collar 38. The sheath 26 serves to secure and cover the stent 48 during delivery thereof. The sheath 26 is axially movable on the shaft 14 of the system 10 so that it can be remotely retracted from over the stent 48, as is known in the art. For example, the sheath 26 may be coupled with a wire pull back system for proximal retraction of the sheath 26 in order to expose the stent 48 for expansion.

Any suitable balloon expandable stent or equivalent known in the art may be used in a stent delivery system in accordance with the present invention. Also, the above description is provided merely to illustrate one example of an inflation-type stent delivery system suitable for use in the present invention, and other now-known or later developed inflation-type stent delivery systems may also be used to form a stent delivery system in accordance with the present invention.

According to the present invention and referring additionally to FIG. 3, the catheter shaft 14 of the stent delivery system 10 defines two lumens 70 and 71 for removably (slidably) receiving a guidewire 80 and a fiber optic cable 81 having an imaging capability, respectively. The guidewire 80 is configured for use in guiding and positioning the stent delivery system 10, as known in the art. Any now-known and later developed guidewire, including any steerable guidewire as known in the art, may be used in a stent delivery system of the present invention. The fiber optic cable 81 has a proximal end and a distal end, and in various embodiments is capable of transmitting illumination light from its proximal end to its distal end while transmitting an image from its distal end to its proximal end. The construction and operation of the fiber optic cable 81 will be more fully described below.

In operation, the guidewire 80 is used to navigate through any tortuous pass into the body cavity or vessel of interest, along which the catheter shaft 14 including the fiber optic cable 81 can follow. Because the fiber optic cable 81 has a viewing capability, a physician can advance the guidewire 80 while observing an image received from the distal end of the fiber optic cable 81. For example, the distal end of the fiber optic cable 81 may be positioned in tandem with the distal end of the guidewire 80 so as to include the distal end of the guidewire 80 within the field of view of the fiber optic cable 81. An image obtained by the fiber optic cable 81 can be used to visually determine the end points of a stricture, and hence the length of the stricture, or to observe tissue and/or lesion in a surrounding area of the stricture, so as to properly position the distal portion of the catheter shaft 14 carrying the stent 48 relative to the stricture to accurately deploy the stent 48 in the stricture.

Once the distal portion of the catheter shaft 14 is positioned in place, the sheath 26 is proximally retracted and the balloon 22 inflated to deploy the stent 48. After the stent 48 is deployed, the catheter shaft 14 is proximally retracted together with the guidewire 80 and the fiber optic cable 81. The fiber optic cable 81 may be used to visually inspect proper deployment of the stent 48 before, during, and after deployment. In some embodiments, at least a portion of the catheter shaft 14 over which the stent 48 is placed is made of clear (transparent) material, so that the fiber optic cable 81 can image the deployment of the stent 48 from within the catheter shaft 14. In alternative embodiments, a mirror, prism, etc. may be selectively arranged relative to the distal end of the fiber optic cable 81 so as to add a backward (or sideways) viewing capability to the fiber optic cable 81. Using these embodiments, the distal end of the fiber optic cable 81 may be placed distal to the distal end of the catheter shaft 14 so as to look back at the stent 48 while it is being deployed.

Additionally, the fiber optic cable may be configured to transmit electromagnetic energy (including both visible and non-visible ranges) for further diagnosis/treatment purposes or imaging in modes other than a white light mode such as fluorescence. For example, based on the fact that cancerous and necrotic tissue has a different density and thus absorbs a different wavelength of light than healthy tissue, the fiber optic cable can be used to irradiate light of a certain wavelength range on the tissue in question, and then to read the light reflected back from the tissue. Suitable software is used to subtract the reflected light from the irradiated light to determine the wavelength of the light that was absorbed by the tissue, thereby making a diagnosis of the tissue.

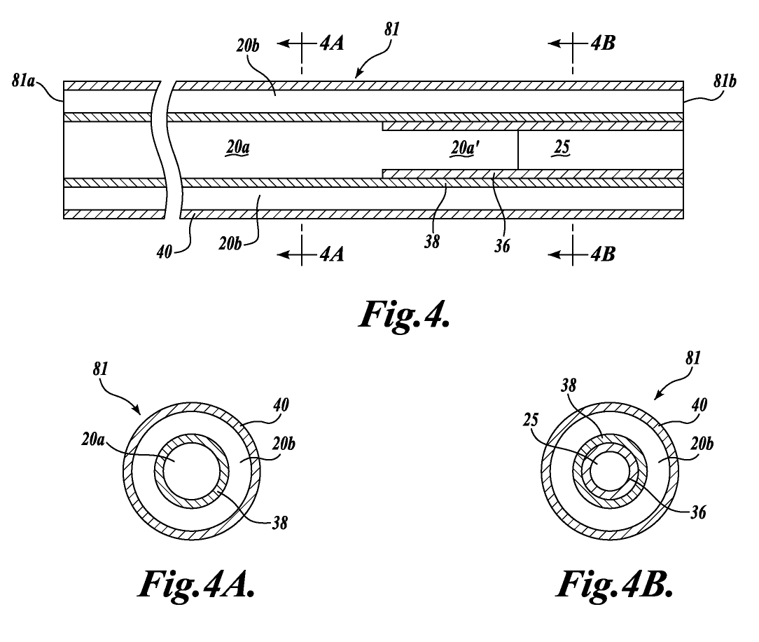

FIGS. 4, 4A, and 4B illustrate one embodiment of a fiber optic cable 81 suitable for use in the present invention. In the illustrated embodiment, the fiber optic cable 81 is configured to transmit illumination light from its proximal end 81a to the distal end 81b, and also to transmit an image from its distal end 81b to the proximal end 81a. In the illustrated embodiment, the fiber optic cable 81 includes one or more centrally extending coherent imaging fibers 20a and one or more circumferentially extending illumination fibers 20b (which may not be coherent) that generally surround the one or more imaging fibers 20a. Further, an objective lens 25 is attached to the distal end of the one or more imaging fibers 20a.

In the illustrated embodiment, the lens 25 and the distal end of the one or more imaging fibers 20a are connected by a transparent adhesive. Further, a non-transparent adhesive is applied on the radially outer surface of the lens 25 and also on the radially outer surface of the distal end portion 20a′ of the one or more imaging fibers 20a, and a first tube 36 is slid thereover to cure the adhesive and to further bond the lens 25 to the distal end of the one or more imaging fibers 20a. Then, a non-transparent adhesive is applied on the radially outer surface of the first tube 36, and a second tube 38 is slid over both the first tube 36 and the one or more imaging fibers 20a. One or more illumination fibers 20b are arranged radially outward of the second tube 38 and are impregnated with a transparent adhesive. A protecting tube 40 is then slid over the impregnated illumination fibers 20b. In one embodiment, the diameter of the lens 25 is 0.35 mm and the overall diameter of the fiber optic cable 20 is 0.78 mm. A suitable fiber optic cable of this type for use in the present invention is available from POLYDIAGNOST GmbH of Germany (www.polydiagnost.com). It should be understood that other types of fiber optic cables having light illumination and image transmission capacities may also be used, as will be apparent to one skilled in the art.

While the illustrated embodiment includes the lens 25 to focus an image for transmission through the one or more imaging fibers 20a, a lens may be omitted in some applications. For example, the distal ends of the one or more imaging fibers 20a themselves may be tapered so as to internally focus an image without an additional lens.

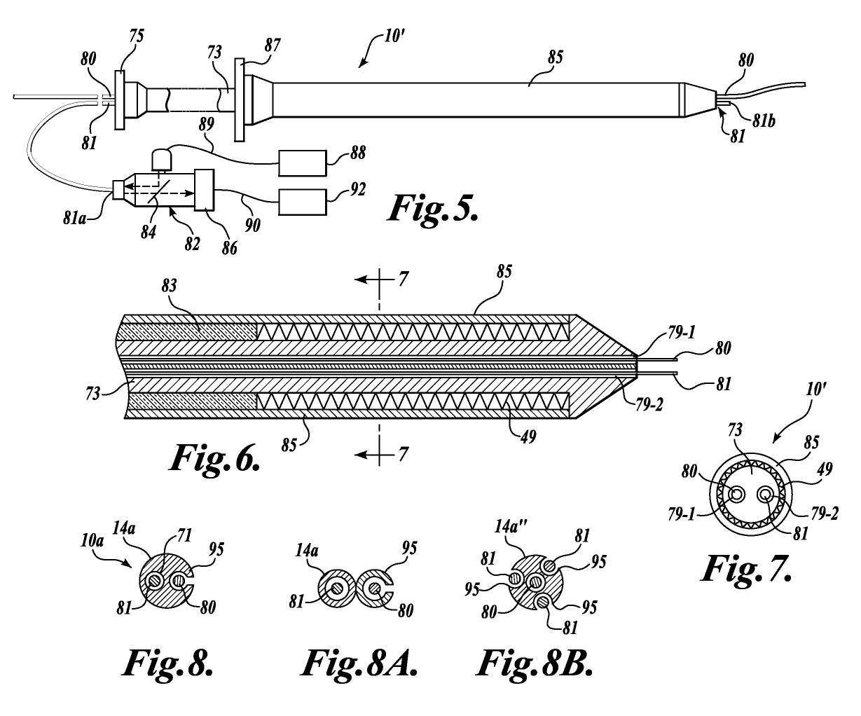

FIGS. 5-7 illustrate a self-expandable type stent delivery system 10′, which is further coupled to an eyepiece 82 for viewing an image received by the fiber optic cable 81. As before, the system 10′ includes a catheter shaft 73 defining two lumens 79-1 and 79-2 for respectively receiving the guidewire 80 and the fiber optic cable 81 therethrough. A handle 75 is provided at the proximal end of the catheter shaft 73. A self-expanding stent 49 is coaxially mounted around the catheter shaft 73 near its distal portion. A space-filling jacket 83 is secured (e.g., by a friction-fit) to the catheter shaft 73 proximally relative to the stent 49 to prevent proximal sliding of the stent 49 during deployment. An outer sleeve 85 is adapted for axial movement relative to the catheter shaft 73 and is coaxially mounted around the self-expanding stent 49 to maintain the stent 49 in a compressed state. A handle 87 is disposed at the proximal end of the sleeve 85 for use in axially moving the sleeve 85 relative to the catheter shaft 73.

Referring specifically to FIG. 5, in the illustrated embodiment, the proximal end 81a of the fiber optic cable 81 is connected to an eyepiece 82. The eyepiece 82 includes a light splitter 84 and a camera or image sensor 86. The light splitter 84 receives illumination light from a light source 88 through a cable 89. The cable 89 may include a group of standard clad optical fibers that function as illumination fibers for carrying the light from the light source 88 to the light splitter 84. The light from the light splitter 84 is coupled to the one or more illumination fibers 20b in the fiber optic cable 81 for delivery to the distal end 81b thereof in order to illuminate the imaged area. An image from the distal end 81b of the fiber optic cable 81 is transmitted through the one or more imaging fibers 20a in the fiber optic cable 81 to the proximal end 81a thereof, and through the light splitter 84 within the eyepiece 82 to the camera or image sensor 86. The image is then processed and supplied from the camera or image sensor 86 via a cable 90 to an image control unit 92 coupled to a display (not shown) that produces an image of the viewed area. Additionally or alternatively, the eyepiece 82 permits direct visualization of the viewed area.

In operation, a physician first introduces the guidewire 80 into the body cavity or vessel of interest, while observing an image received from the fiber optic cable 81 via the eyepiece 82. The catheter shaft 73 then follows the guidewire 80 and the fiber optic cable 81, both of which are removably received within its two lumens 79-1 and 79-2, respectively. Once the distal portion of the catheter shaft 73 is properly positioned, the outer sleeve 85 is proximally retracted so as to permit the stent 49 to expand. After the stent 49 is deployed, the catheter shaft 73 including the guidewire 80 and the fiber optic cable 81 is proximally retracted. As before, the fiber optic cable 81 may be used to observe proper deployment of the stent 49 before, during, and after deployment.

Any suitable self-expanding stent or equivalent known in the art may be used in a stent delivery system in accordance with the present invention. Furthermore, the above description merely illustrates one example of a self-expanding type stent delivery system suitable for use in the present invention, and other now-known or later developed self-expanding type stent delivery systems may also be used to form a stent delivery system in accordance with the present invention.

While in the above described embodiments, the fiber optic cable 81 is illustrated as being removably (slidably) received within one of the lumens in the catheter shaft. However, the fiber optic cable 81 may be non-removably received within a catheter lumen in some applications. For example, in some applications it may be desired to fix a distal end of the fiber optic cable 81 (i.e., the image acquisition point) relative to the catheter shaft during delivery and deployment of a stent. This may be accomplished, for example, by integrally forming the fiber optic cable 81 with the catheter shaft during the extrusion process, by over-extruding a plastic material over the fiber optic cable 81. Alternatively, the fiber optic cable 81 may be fixed to the catheter shaft by means of adhesive, by using a shrink-fit method, etc.

In some embodiments of the present invention, a catheter shaft may define further lumens, in addition to the two lumens for receiving the guidewire 80 and the fiber optic cable 81, to receive various other medical catheters/equipment or to transport liquids or gasses for use in various surgical operations.

Referring to FIG. 8 and in accordance with another embodiment of the present invention, a stent delivery system 10a includes a catheter shaft 14a defining one lumen 71 for removably receiving a fiber optic cable 81 therethrough. In this embodiment, the catheter shaft 14a further defines a guide 95 having a generally C-shaped (or U-shaped) cross-section. The guide 95 serves to contain, but not necessarily constrain, a guidewire 80. A catheter including a guide (or channel) similar to the guide 95, which permits easy radial access to the guidewire 80 from a location exterior to the catheter shaft, is known in the art as a rapid exchange catheter, as described in U.S. Pat. No. 6,007,522, which is incorporated by reference herein. Briefly, a rapid exchange catheter permits the use of a relatively shorter guidewire, and also the rapid exchanging of different catheters/devices used during a medical procedure. In various embodiments, the overall cross-sectional shape of the catheter shaft 14a is generally circular, as illustrated in FIG. 8, to permit smooth movement of the catheter shaft 14a within a patient's body cavity or vessel, though the cross-sectional shape of the catheter shaft 14a is not so limited. For example, in other embodiments as shown in FIG. 8A, a guide 95 may be provided externally along the side of the catheter shaft 14a, so that the catheter shaft 14a and the guide 95 provided in a side-by-side manner together form a generally “FIG. 8” cross-sectional shape. The guide 95 may extend axially along at least a portion of the axial length of the catheter shaft 14a without interfering with the proper operation of the stent delivery system 10a (e.g., the deployment of the stent). Alternatively, the guidewire 80 may be received within the guide 95, and the fiber optic cable 81 may be received within the guide 95 to permit easy radial access to the fiber optic cable 81 from a location exterior to the catheter shaft 14a. In this embodiment, the catheter shaft 14a includes a lumen through which the guidewire 80 extends. Further alternatively, while only one guide 95 may be provided, plural guides 95 may be provided, as shown in FIG. 8B, in a spaced apart manner around the circumference of the catheter shaft 14a to respectively receive plural fiber optic cables 81 (or plural guidewires) therein.

In operation, as before, the guidewire 80 is used to first reach the location of interest within the body cavity or vessel, after which the catheter shaft 14a and the fiber optic cable 81 can follow. A physician can adjustably position the distal end of the fiber optic cable 81 that is slidably received within the lumen 71 (or the guide 95) relative to the distal end of the guidewire 80 so as to observe an image received from the distal end of the fiber optic cable 81 to assist in properly advancing the guidewire 80. As before, an image obtained by the fiber optic cable 81 can be used to determine both the end points and the length of a stricture, or to observe an area surrounding to stricture, to properly position the distal portion of the catheter shaft 14a carrying a stent relative to the stricture to accurately deploy the stent in the stricture. After the stent is deployed, the catheter shaft 14a is proximally retracted together with the guidewire 80 and the fiber optic cable 81. As before, the fiber optic cable 81 may be used to visually inspect proper deployment of the stent before, during, and after deployment.

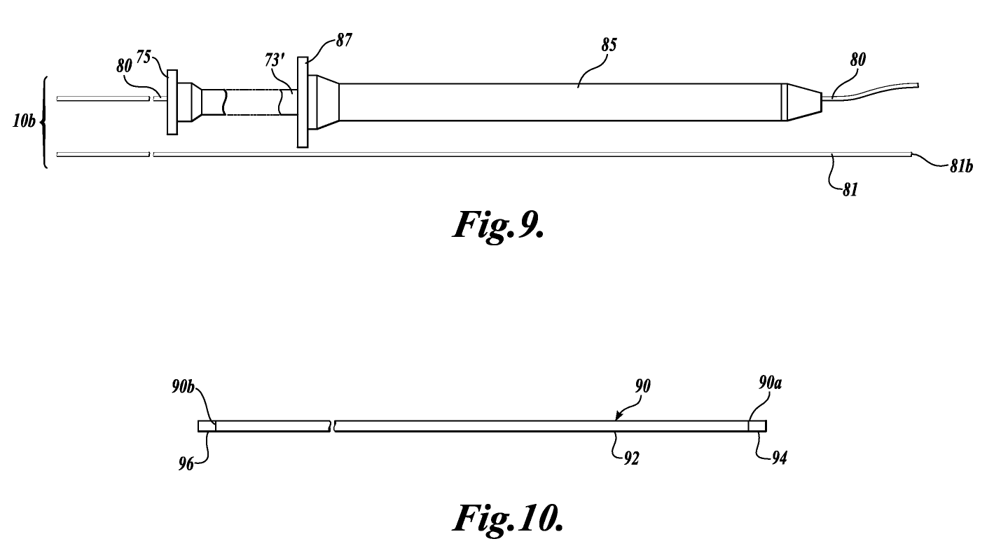

Referring to FIG. 9 and in accordance with an alternative embodiment of the present invention, a stent delivery system 10b includes a catheter shaft 73′, and the stent delivery system 10b is of self-expandable type similarly to the embodiment shown in FIG. 5, described above. It should be understood, however, that the present embodiment may be realized in a stent delivery system of inflation type also, and the self-expandable type illustrated in FIG. 9 is provided merely as an example. Unlike the catheter shaft 73 of FIG. 5, the catheter shaft 73′ of FIG. 9 defines one lumen for removably receiving a guidewire 80 therethrough. The stent delivery system 10b further includes a fiber optic cable 81, which is provided independently of (or outside) the catheter shaft 73′.

In operation, as before, the guidewire 80 is used to first reach the area of interest in the body cavity or vessel, after which the catheter shaft 73′ can follow. A physician can advance the guidewire 80 in a generally side-by-side manner with the fiber optic cable 81. For example, a physician can adjustably position the distal end of the fiber optic cable 81 relative to the distal end of the guidewire 80 so as to observe an image received from the distal end of the fiber optic cable 81 to assist in properly advancing the guidewire 80. As before, an image obtained by the fiber optic cable 81 can be used to determine the proper position at which the stent is to be deployed. After the stent is deployed, the catheter shaft 73′, which includes the guidewire 80, and the fiber optic cable 81 are both proximally retracted. The fiber optic cable 81 may again be used to visually inspect proper deployment of the stent before, during, and after the deployment.

According to the present invention, various embodiments of a stent delivery system are provided, which are adapted to accommodate both a guidewire and a fiber optic cable having an imaging capability. The use of a fiber optic cable with an imaging capability permits a physician to visually observe not only the proper advancement of the guidewire but also the proper deployment of a stent. Thus, the present invention provides a compact stent delivery system, which reduces the need to rely on fluoroscopy or a relatively larger-diameter endoscope to deliver and deploy a stent. The stent delivery system of the present invention is suited for delivering and deploying a stent in various systems in a patient including GI (gastrointestinal), URO (urogenital), biliary, and vascular systems.

While the preferred embodiments of the invention have been illustrated and described, it will be appreciated that various changes can be made therein without departing from the spirit and scope of the invention. For example, while the present invention has been described as using a fiber optic cable for illuminating and imaging an object before, during, and after the deployment of a stent, in alternative embodiments, an image sensor provided at a distal end of a signal cable may be used in place of a fiber optic cable for imaging an object. Specifically, referring to FIG. 10, an elongate imaging device 90 for use in place of a fiber optic cable consists of a flexible signal cable 92 having a distal end 90a and a proximal end 90b. An image sensor 94 is provided at the distal end 90a while an electrical connector 96 is provided at the proximal end 90b of the signal cable 92. The image sensor 94 may be a CCD, CMOS, pin hole, photo diode, or any other type of sensor. An image obtained by the image sensor 94 is transmitted via the signal cable 92 to its proximal end and to the electrical connector 96, which provides electrical connections to an image processor (not shown) such that the image from the image sensor 94 can be received and processed. The image sensor 94 may be made movable to provide both forward and rearward viewing capabilities. The use of the imaging device 90 consisting of a signal cable and an image sensor in a stent delivery system of the present invention is the same as that of the fiber optic cable, described above.

The embodiments of the invention in which an exclusive property or privilege is claimed are defined as follows:

1. A stent delivery system, comprising:
a catheter shaft defining first and second lumens therein;
a stent positioned over the catheter shaft;
a guidewire removably received within the first lumen, wherein the guidewire is configured to be independently advanced from the catheter shaft; and
a fiber optic cable having a first end and a second end, the fiber optic cable transmitting illumination light from its first end to its second end while transmitting an image from its second end to its first end, the fiber optic cable being removably received within the first lumen and configured to be independently advanced from the catheter shaft such that the fiber optic cable views the stent.

2. The system of claim 1, wherein the fiber optic cable is configured to view the stent before, during, and after a stent deployment procedure.

3. The system of claim 1, wherein the stent is of self-expanding type, further comprising a proximally retractable sleeve coaxially placed over the stent for deploying the stent.

4. The system of claim 1, wherein the stent is of inflation type, further comprising an inflatable balloon positioned between the catheter shaft and the stent for deploying the stent.

5. The system of claim 1, wherein at least a portion of the catheter shaft is made of clear material so as to permit viewing of the stent with the fiber optic cable through the clear portion of the catheter shaft when the second end of the fiber optic cable is within the catheter shaft.

6. The system of claim 1, wherein the fiber optic cable further comprises an optical element placed at its second end to achieve a backward viewing capability.

7. A method of delivering and deploying a stent within a body cavity or vessel, comprising:
(a) providing a stent delivery system, comprising:
(i) a catheter shaft defining first and second lumens therein;
(ii) a stent positioned over the catheter shaft;
(iii) a guidewire removably received within the first lumen; and
(iv) a fiber optic cable having a first end and a second end, the fiber optic cable transmitting illumination light from its first end to its second end while
transmitting an image from its second end to its first end, the fiber optic cable being removably received within the second lumen;
(b) advancing the guidewire through the body cavity or vessel to a desired position;
(c) passing the catheter shaft along the guidewire;
(d) advancing the fiber optic cable through the body cavity or vessel, such that the fiber optic cable views advancement and deployment of the stent;
(e) deploying the stent.

8. The method of claim 7, further comprising, with the fiber optic cable, observing an image of the stent after its deployment.

9. The method of claim 7, further comprising removing a cover from the stent to allow it to self-expand at the desired position.

10. The method of claim 7, further comprising deploying the stent by inflating a balloon beneath the stent.

11. A stent delivery system, comprising:
a catheter shaft defining a lumen therein, the catheter shaft further defining a guide which extends axially along at least a portion of an axial length of the catheter shaft;
a stent positioned over the catheter shaft;
a guidewire removably received within the guide, wherein the guidewire is configured to be independently advanced from the catheter shaft; and
a fiber optic cable having a first end and a second end, the fiber optic cable transmitting illumination light from its first end to its second end while transmitting an image from its second end to its first end, the fiber optic cable being removably received within the lumen and configured to be independently advanced from the catheter shaft such that the fiber optic cable views the stent.

12. The system of claim 11, wherein the fiber optic cable is configured to view the stent before, during, and after a stent deployment procedure.

13. The system of claim 11, wherein at least a portion of the catheter shaft is made of clear material so as to permit viewing of the stent positioned over the catheter shaft with the fiber optic cable through the clear portion of the catheter shaft when the second end of the fiber optic cable is within the catheter shaft.

14. The system of claim 11, wherein the fiber optic cable further comprises an optical element placed at its second end to achieve a backward viewing capability.

15. The system of claim 11, wherein the guide has a generally circular-shaped cross section

16. The system of claim 11, wherein the guide has a generally C-shaped cross section.

17. A method of delivering and deploying a stent within a body cavity or vessel, comprising:
(a) providing a stent delivery system, comprising:
(i) a catheter shaft defining at least one lumen therein;
(ii) a guidewire removably received within the lumen;
(iii) a stent positioned over the catheter shaft; and
(iv) a fiber optic cable having a first end and a second end, the fiber optic cable transmitting illumination light from its first end to its second end while transmitting an image from its second end to its first end;
(b) advancing the guidewire through the body cavity or vessel to a desired position;
(c) passing the catheter shaft along the guidewire;
(d) advancing the fiber optic cable through the body cavity or vessel, such that the fiber optic cable views advancement and deployment of the stent; and
(e) deploying the stent.

18. The method of claim 17, further comprising, with the fiber optic cable, observing an image of the stent after its deployment.

19. A stent delivery system, comprising:
a catheter shaft defining first and second lumens therein;
a stent positioned over the catheter shaft;
a flexible guidewire removably received within the first lumen, wherein the flexible guidewire is configured to be independently advanced from the catheter shaft; and
an elongate imaging device being removably received within the second lumen, the device having a first end and a second end, the device transmitting an image from its second end to its first end, the elongate imaging device being configured to be independently advanced from the catheter shaft such that the elongate imaging device views the stent.

20. The system of claim 19, wherein the elongate imaging device is configured to view the stent before, during, and after a stent deployment procedure.

21. The system of claim 19, wherein the elongate imaging device comprises a fiber optic cable having a first end and a second end, the fiber optic cable transmitting illumination light from its first end to its second end while transmitting an image from its second end to its first end.

22. The system of claim 19, wherein the elongate imaging device comprises a signal cable having a proximal end and a distal end and an image sensor coupled to the distal end of the signal cable, the signal cable transmitting an image obtained by the image sensor from its distal end to its proximal end.

23. The system of claim 19, wherein at least a portion of the catheter shaft is made of clear material so as to permit viewing of the stent with the elongate imaging device through the clear portion of the catheter shaft when the second end of the elongate imaging device is within the catheter shaft.

24. The system of claim 19, wherein the elongate imaging device further comprises an optical element placed at its second end to achieve a backward viewing capability.
